(19) United States
(12) Patent Application Publication
(10) Pub. No.: US 2023/0057972 A1
(43) Pub. Date: Feb. 23, 2023

(54) SYSTEM AND METHOD FOR CLASSIFYING SENSOR READINGS

(71) Applicant: UTComp Inc., (US)

(72) Inventor: Geoffrey CLARKSON, Cambridge, CA (US)

(21) Appl. No.: 17/976,452

(22) Filed: Oct. 28, 2022

Related U.S. Application Data
(63) Continuation of application No. PCT/CA2021/050599, filed on Apr. 30, 2021.

(51) Int. Cl. G06F 17/40; G06K 9/62

(52) U.S. Cl. CPC G06F 17/40; G06K 9/628; G06K 9/6262

(57) ABSTRACT

A system and method to evaluate and/or classify non-destructive testing sensor data, the system and method including: a transmitter configured to provide energy to a material; one or more sensors configured to convert the energy returned from the material into sensor data; a receiver configured to receive sensor data; an attenuation inversion module configured to apply a mathematical transformation to the sensor data to provide transformed sensor data; an analysis module configured to process the transformed sensor data to provided processed sensor date, by: determining values from the transformed sensor data; applying mathematical transformations to the values to produce a set of single values that represent the sensor data; a classification module configured to classify the processed sensor data; and an output module configured to output the results of the classification.

### RELATED APPLICATIONS

This application is a continuation of PCT App. No. PCT/CA2021/050599, filed Apr. 30, 2021, which claims priority to U.S. Provisional Patent Application No. 63/017,823 filed Apr. 30, 2020, both of which are hereby incorporated herein in their entirety.

### FIELD

This application relates to material testing and more particularly to a system and method for classification, categorization, evaluation or sorting of sensor readings used to provide information on materials or components.

### BACKGROUND

Traditionally, various non-destructive testing methods are used to evaluate materials or components for the existence of features, properties or defects (“information”) that are related to the ability of the material or component to function as intended by its design. These non-destructive evaluations can take place at various times in the usage cycle of the material or component: for example; at times during manufacture, immediately after manufacturing, after the material or component has been in service for a period of time, or at other times.

Conventionally, when using non-destructive testing methods, energy or force is often applied to the material or component. This energy or force (input) is transformed by the material or component and the transformed energy is received by, for example, sensors that convert the transformed energy into data that can be evaluated. Various equipment has been commercialized and is in common use to apply the energy and to receive the transformed energy. Generally, accepted practice is for the transformed energy data to be presented on a visual display for evaluation. The visual display is then viewed by personnel who are trained to interpret the transformed energy data or signals and assess whether specific features or patterns exist within the data. The personnel may then compare the features or patterns with criteria that classify the material or component—for example if it is “Acceptable” or “Not Acceptable”.

Trained personnel can be difficult to find and such personnel may not have experience with all types of situations. As such, additional personnel may need to be involved. Further, there can be issues with time delays associated with additional evaluation efforts; variation that is introduced by subjective opinion; misinterpretation os false signals; and other issues as they may occur.

Embodiments of the system and method described herein are intended to address at least one of the issues with analysis, interpretation and evaluation of transformed energy data from sensors used for non-destructive testing of materials and components.

### SUMMARY

In a first aspect, the present disclosure provides a system to evaluate/classify non-destructive testing sensor data, the system including: a transmitter configured to provide energy to a material; one or more sensors configured to convert the energy returned from the material into sensor data; a receiver configured to receive sensor data; an attenuation inversion module configured to apply a mathematical transformation to the sensor data to provide transformed sensor data; an analysis module configured to process the transformed sensor data to provided processed sensor date, by: determining values from the transformed sensor data; applying mathematical transformations to the values to produce a set of single values that represent the sensor data; a classification module configured to classify the processed sensor data; and an output module configured to output the results of the classification.

In some cases, the system may further include a data storage module configured to store the values determined from the processing module and the results of classification.

In some cases, the system may further include a memory component configured to store the parameters to be used in the mathematical transformations used in the attenuation inversion module.

In some cases, the system may further include a data storage module configured to store a distribution of known classes for the sensor data; the values determined from the processing module; the results of classification; and specific values for limits and boundaries on values determined from the signal.

In some cases, the configuration data may include data associated with the estimated physical dimensions of the material or component.

In some cases, the system may further include a calculation component that is configured to determine parameters and transformations to define new classes.

In some cases, starting and ending coordinates may be applied to the sensor data to be evaluated.

In some cases, the data may be associated with the energy transmitter, the at least one sensor, and the receiver is included with the sensor data.

In some cases, starting and ending coordinates may be applied to the sensor data to be evaluated as a value for the boundaries determined from the sensor.

In some cases, the classification module may be configured to determine the most probable class related to sensor data.

In some cases, the classification module may be configured to identify data that is to be stored in the data storage module.

In some cases, the classification module may be configured to provide data relating to the transformation of values calculated from the sensor data.

In some cases, the output module may be configured to provide data related to whether sensor data is within a stated confidence interval for its classification.

In some cases, the data storage module may be configured to include specific values for limits and boundaries on values determined from signal data.

In some cases, the data storage module may be configured to include data relating to the transformation of values calculated from signal data to form a single characteristic value for a set of signal data.

In some cases, the data storage module may be configured to contain data relating to the distribution of characteristic values relative to data classifications.

In some cases, the classifier module may be configured to calculate data relating to the transformation of values calculated from signal data.

In some cases, the classifier module may be configured to provide data relating to the transformation of values calculated from signal data. The data relating to the transformation may be stored by the data storage module.

In some cases, the output module may be configured to provide data in a digital format for use by computer programs.

In another aspect, the present disclosure provides a method to evaluate and classify sensor data, the method including: applying energy to a material; receiving transformed energy at sensors as signal data; amplifying the signal data, in an attenuation inversion module; processing the amplified signal data, at a signal processing module; classifying processed signal data; storing processed signal data related to the sensor signals in a data storage module together with the results of classification; displaying the classification results, at an output module; transferring stored data to a classifier module, wherein the data is used to calculate and update transformations and distributions to be applied to future data; and storing the updated transformations and distributions in a data storage module.

In some cases, the method may further include storing a plurality of possible classifications of the sensor data.

In some cases, the attenuation inversion calculations may be applied to the sensor data.

In some cases, the sensor data may include estimated physical dimensions of the material.

In some cases, the method may further include applying limits and boundaries on values determined from the signal data and provided by the data storage module.

In some cases, the method may further include using stored values for limits and boundaries on values determined from signal data and using starting and ending coordinates in the data set for the signal data to be evaluated.

In some cases, the method may further include transforming the signal data into characteristic values using specific data provided by the data storage module.

In some cases, the method may further include comparing characteristic values from transformation of the signal data to a known distribution.

In some cases, the method may further include providing output related to the most probable class related to the sensor data.

In some cases, the sensor data from materials that are confirmed to be reference samples may be used to develop new transformations to be used in classification.

In some cases, a new distribution of results may be calculated based on the new transformations.

In some cases, the configuration data may include data associated with the energy transmitter, sensors, and receiver.

In some cases, the method may include classifying the signal data based on calculation of the probability that it is a member of a particular class.

In some cases, the method may include providing the output module with data in a digital format for use by computer programs.

In some cases, the method may include providing for the new transformations and distribution information to be stored in the data storage module.

Other aspects and features of the present disclosure will become apparent to those ordinarily skilled in the art upon review of the following description of specific embodiments in conjunction with the accompanying figures.

### BRIEF DESCRIPTION OF FIGURES

Embodiments will now be described, by way of example only, with reference to the attached drawings, in which:

FIG. 1 illustrates a system for classification of sensor data according to an embodiment;

FIG. 2 illustrates an example of applying starting and ending coordinates to time-based sensor data;

FIG. 3 illustrates an example of applying starting and ending coordinates to spatial sensor data;

FIG. 4 is a graphical representation of the desired appearance of the sensor data after the attenuation inversion;

FIG. 5 is an illustration of the distribution of scalar values calculated from reference data;

FIG. 6 illustrates a graphical representation of one-dimensional sensor data as it appears directly from the sensors at the receiver module;

FIG. 7 is a graphical representation of one-dimensional sensor data after the attenuation inversion module with example values that can be determined from the data;

FIGS. 8A and 81 illustrate two different classes on one-dimensional sensor data;

FIGS. 9A and 9B illustrate two-dimensional sensor data; and

FIG. 10 illustrates a flowchart of a method for classification of sensor data according to an embodiment.

### DETAILED DESCRIPTION

Generally, there is provided a system and method for classification/evaluation (sometimes referred to as categorization or sorting) of sensor data, generally non-destructive sensor data, taken from materials for the purpose of determining various features and properties of the materials being tested. An embodiment of the system may generally include: an energy source such as a transducer configured to apply energy to a material, sensors configured to detect and convert energy related to the applied energy in various locations into sensor signals/data, a receiver configured to collect and store the sensor data, a filter module configured to filter the sensor data, a signal processing module configured to process the filtered sensor data, an analysis module configured to analyze the processed sensor data by: calculating a number of characteristic values from the sensor data; comparing the calculated characteristic values with reference values; correlation of characteristic values to a known distribution; classification of the signal data; determining if the signal data can provide a valid solution to evaluation; and an output module configured to output the results of the determining as a signal evaluation.

In this application, the following general definitions will be used:
Data: A set of numerical values that represent a sensor signal.
Class: A description or template of data that describes certain patterns or attributes. As most non-destructive testing results tend to be displayed graphically, typical classes for this would include items such as the appearance of results with no defects, the appearance of particular defects, and the like.
Transformation: A mathematical operation that can be used to convert data into a single value or a set of values. Transformations could take several forms and in this description they generally convert several data values into a smaller number of values.
Parameter: A numerical value that is calculated from data using transformations or mathematical operations. In the context of this system and method, parameters are intended to reflect the reduction of data to determine if the data produces a parameter result that is similar to a class.
Definition values: Values that are used to define the values of parameters for use in classification. Parameters calculated from data are often compared to these definition values in the screening process.

It will be understood that when the signal data contains very clear features or patterns, identification and classification may be more direct/basic. These situations may be more common in quality control situations, for example in a controlled manufacturing environment or the like.

In other circumstances, the materials or components may not immediately yield signal data that provides for a direct/basic interpretation of the required features or patterns at the time of inspection such that there is a barrier to classification. There may be several circumstances that exist to create this barrier. Some examples of barriers include: when the signal data also includes a large amount of noise, such as other signals, reflected signals, environmental effects, and the like the nature of the material or the component obscures the energy transformation, thereby obscuring the features and patterns within the signal data; energy transfer into the material or component may be flawed or impaired; operator error by the personnel gathering the signal data; or the like.

In these circumstances where interpretation of the transformed energy/signal data may be more complex, it may be desirable to determine if the signal data obtained is useful in evaluation. Data that can be usable in evaluation may include, for example; patterns and values that are clearly different from the applied signal, thus providing evidence that the signal was transformed by the material or component; data that includes patterns or values that are known to be relevant to the material or component being tested; or other information related to the signal data.

In some cases, data obtained might not be usable and this determination by the system will allow other attempts to obtain usable data in a timely manner. This determination may also allow some immediate decisions regarding disposition of a material or component.

Examples where data is not useable may include: situations where no signal is detected at the receiver that could have been transmitted from the transmitter; situations where the signal received has not been transformed by the material or component thereby showing that the transmitted signal did not enter the material or component; situations where the signal provided by the receiver only contains values of zero (0); situations where the data in the signals received does not comply with specifications for instrumentation configuration; situations where the signals received are known to not include the data that may be of interest for evaluation; and other reasons.

Experience with testing and inspection of systems of materials and components has shown that evaluation of certain characteristics of the materials or components is possible using techniques and tools that gather information without damaging the tested material. There are several techniques for non-destructive testing where energy that is applied to the material is transformed and subsequently received by the sensor. Some examples of these techniques include: ultrasonic testing, radiography, eddy current testing, magnetic particle testing, thermography, and others. Some of these tests provide unambiguous, quantitative results that are generally considered to be calibrated measurements.

Each non-destructive test method may have particular characteristics that make the method better suited for some types and configurations of material. In general, a method is selected and used based on the expertise of the personnel performing the test. Any non-destructive testing method may benefit from embodiments of the system and method described herein, in particular, those that produce a quantitative data output based on measurements.

Moreover, since non-destructive methods do not affect the usability of the material or component, these techniques and tools can be used to monitor the materials and components both before use and for changes that may occur after use.

Conventionally, non-destructive testing techniques have been developed to provide a representation that can be related to the physical configuration of the item being tested for interpretation of the results.

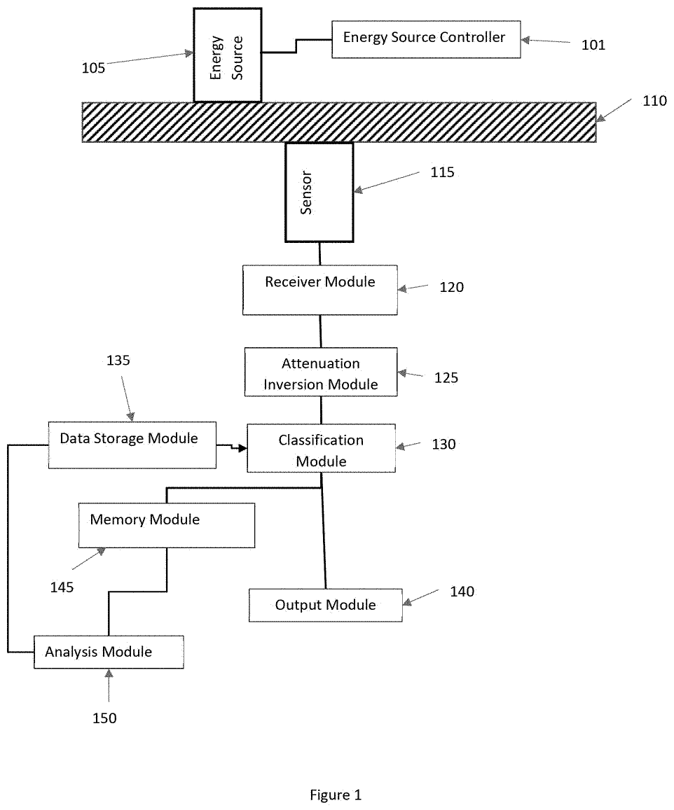

FIG. 1 illustrates a system for classifying sensor data acquired from application of non-destructive energy to a material or component that is being tested. The figure illustrates a general configuration of a material or component. The material or component may be or be formed from, for example, steel, aluminum, polymer, reinforced polymer, or other materials. The non-destructive test method may include ultrasonic testing, radiography, eddy current testing, conductivity testing, thermography or other method. In most cases, these test methods apply energy to the material in the same form as the sensors receive the energy. It will be noted that there may be circumstances where the energy applied to the material may be in a different form than the energy received by the sensor. For example: friction from mechanical energy can increase surface temperature that is detected by thermography; fluid flow over a surface can create vibrations detected by ultrasonic sensors; electrical current in a wire produces a magnetic field that is detected using magnetic sensors; and other situations of this type.

In the system shown in FIG. 1, energy source controller 101 is used to control the energy source 105 that applies or transmits energy to the material or component that is being tested 110. At least one sensor 115 detects energy that has been transformed by 110 and converts the energy to a signal that is sent to the receiver module 120. In some cases, there may be a single sensor. In other cases, there may be a plurality of sensors detecting the energy. In some cases, the at least one sensor may send electrical signals as the signal sent to the receiver module 120. In some cases, data associated with the energy transmitter, the at least one sensor and the receiver module may be included in the sensor data.

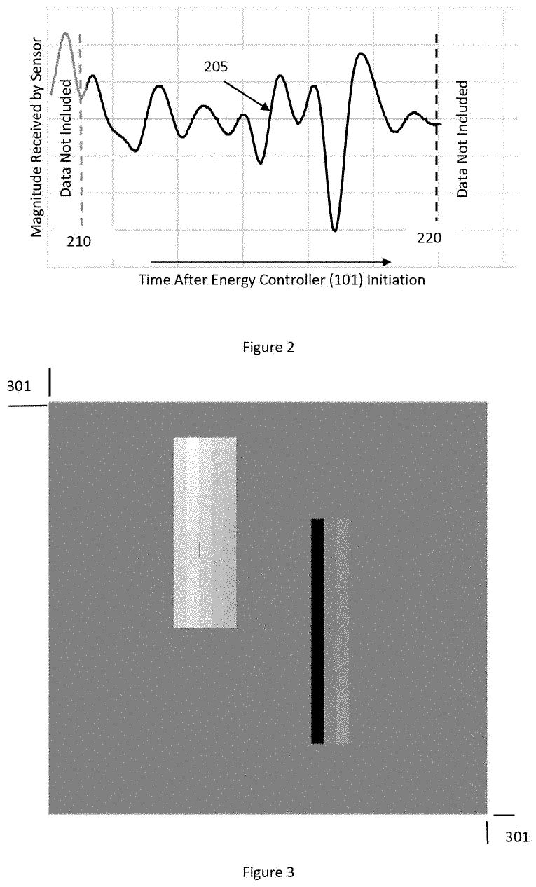

In some further cases, starting and ending coordinates may be applied to the signal data to be evaluated. As an example, there may be a time delay between initiation of the energy application by the energy source controller 101 and application of the energy to the material 110, and a time delay corresponding to the transmission of the energy through the material. FIG. 2 illustrates an example of this situation with a graph showing the received signal 205 as a magnitude at a time after energy controller initiation. In this example, a starting coordinate 210 corresponds to time after energy application and an ending coordinate 220 is time after energy application and allowance for all energy transmission through the material. Various other times may also be defined.

FIG. 3 illustrates an example of spatial starting and ending coordinates. This data is intended to provide a 3 dimensional image where the information to be evaluated is contained within the boundaries of the image. The shading in the image represents the magnitude of the signal received. The starting and ending coordinates for the image may be represented by boundaries 301 of the image and, in some cases, data beyond the boundaries of the image may not be evaluated.

Returning to FIG. 1, the sensor data from the receiving module 120 passes to the attenuation inversion module 125 where portions of the signals that may be attenuated or reduced due to factors such as: distance from the energy source; interference from ambient conditions; signal interruption by an interface; or other causes, can be corrected/inverted. The correction is intended to provide clearer identification of data patterns and to equalize the contribution of all data that has been received to the classification.

Attenuation inversion is intended to correspond to the attenuation of energy that normally occurs as, for example, the energy traverses undamaged media. In the case of ultrasonic signals in material, attenuation corresponds to an equation of the form:

$$Ultrasonic Attenuation = \frac{1}{{e}^{constant \times time}}$$

where the constant in the equation can be determined from historical values for undamaged materials. In the case of radiation such as light, attenuation corresponds to an equation of the form:

$$Radiation Attenuation = \frac{1}{Distance from {Source}^{2}}$$

Attenuation of other types of energy, such as magnetic fields can also be inverted. These inversions are intended to be applied in the attenuation inversion module 125 to provide more robust inverted data.

The results, sometimes called inversion data, are then conducted to the classification module 130. The classification module 130 can use definition values for parameters supplied by the data storage module 135. In some cases, the parameters may include physical dimensions of the material or component being tested. Examples of other parameters and typical transformations are detailed herein. The classification module may be configured to determine data relating to the transformation of values received from the signal data and may be configured to provide this classification data to, for example, the output module or analysis module.

The attenuation inversion module 120 may also be used to apply adjustments to the signal data that are related to effects such as: distance from the energy source to the sensor; distance from the energy source to a region of interest; existence of items that may block the energy or place the item or region of interest into, for example, a shadow; absorption of energy within its path; diffusion of energy away from its path; and others as will be understood depending on the application of the technology.

In some cases, the system may further include an analysis module 150, an output module 140 and a memory module 145. The analysis module 150 may be configured to perform calculations on the signal, inverted or classification data retrieved from the memory module 145. The analysis module 150 may apply transformations to the data to produce a set of parameters that may be used to represent the collected sensor data.

The memory module 145 is configured to store processed data from the analysis module 150. In some cases, the memory module 145 may be further configured to store the parameters used by the attenuation inversion module 125 and the transformations used in the analysis module 150. In some cases, the data storage module 135 or the memory module 145 may be further configured to store the distribution of known classes for sensor data.

The data storage module 135 may be configured to include specific definition values for limits and boundaries or to define the distribution of the values using mean, standard deviation, distribution type, and the like for parameters determined from the signal data. The data storage module 135 may also be configured to include data and parameters that can be used to modify the transformation to improve the results of values determined from the signal data to form a single characteristic value. Examples include retaining parameters determined from sensor results that are shown to fit a certain class so they can be used to modify existing transformations and defining values, which is intended to improve future accuracy. Further description of the overall method is provided herein with example parameters. For example, one example indicates how parameters A_{B}, t_{c}, and I2Disp are transformed into parameter D_{Reference}. When particular data is identified as a good example of a class of data, the relevant parameters will be stored in data storage module 135 so they can be used to improve the transformations and defining values used.

In some cases, the system may be located in one physical location. In other cases, the system may be distributed and, for example, the analysis module 150 may be located separately and may be operatively connected to the data storage module and the classification module. The memory module may be configured to provide a shorter term memory component to the system for data to be transferred to the analysis module or may be remote such as in the cloud or the like. The output module 140 can be configured to provide the output of the classifications of the data to a user of the system or the like. In some cases the classification may provide a probable class related to the sensor data which may be the output provided by the output module 140. In some cases, the classification module 130 and analysis module 150 may determine a confidence interval with respect to the classification and the output module 140 may provide the confidence interval for the classification as part of the output. The output provided by the output module 140 is intended to be in a format readable by a computer program that is configured to receive the results and use them or report on them.

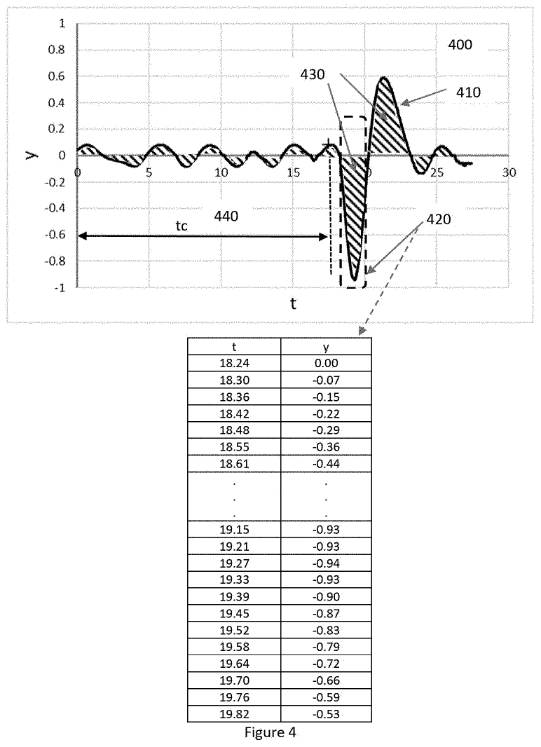

FIG. 4 shows a graphical example of data output 400 from the attenuation inversion module 125 for a sample of material 110 that has been identified to be a reference for samples to be tested—sometimes referred to as a reference sample. In this example, the sample relates to an ultrasonic test where ultrasonic energy (ultrasonic signal or vibrations) have been applied to the material 110. This output 400 may have been determined as a result of specifications or other analysis. The locus 410 is a graphical representation of a two-dimensional array of data values. The data values corresponding to the portion of the locus 410 in the area outlined by the rectangular zone 420 are listed as a table of “t” and “y” values in FIG. 4. In a conventional evaluation and classification practice, one who is skilled in the art would compare a displayed locus image of sensor data from a material sample to the image 400 and determine if they are sufficiently similar in appearance.

In this example, it may be determined that three quantities can serve to represent the graphical data. These quantities represent characteristics that may be directly related to the transformation of the applied energy by the material 110. These quantities are labeled as characteristics. The first is the area of cross-hatching (430) that corresponds to the areas bounded by the bold locus (410) and line at y=0, in this case, the value is the sum of fourteen sub-areas shown in the image. FIG. 4 further illustrates tc (440), which is the horizontal coordinate of the area centroid; I2Disp as the second moment of area of the cross-hatched areas about tc (440). The quantities are calculated directly from the data provided in the tabulated form used to generate the data output 400. Hereafter, these values are identified as: A_{B} for the area bounded; tc for the horizontal coordinate of the centroid of the area bounded; and I2Disp for the second moment of the bounded areas. It will be understood that the calculations and the number of quantities used may vary depending on the testing and the desired results.

When a plurality of reference specimens or samples are used, some variation may be expected in the appearance of the data output 400 from materials or components 110, with corresponding variation of the values for: A_{B}; tc; and I2Disp. Values from several examples of the data output 400 for these reference specimens of material or components may be assembled to produce a model of the distribution of inter-relationships of the values that should be expected. It will be understood that there may be more or fewer values and there may be more or fewer samples.

From the data provided by the available samples of desired data outputs 400, values are calculated as follows by the analysis module for this example with three values. For all of the reference or ideal samples used, the average value of each value A_{B}, tc, and I2Disp is calculated as A_{B}, Atc, and AI2Disp. In this example, there are six possible ways that the three values can be combined into pairs that can be compared: A_{B} with A_{B}; A_{B} with tc; A_{B} with I2Disp; tc with tc; tc with I2Disp; and I2Disp with I2Disp. For each of these combinations, equation 1 is calculated by the analysis module 150:

$${s}_{a , b} = \frac{1}{N} {\sum }_{1}^{N} ( ( Value of a - {A}_{a} ) ( Value of b - {A}_{b} ) ) \qquad ( 1 )$$

### Where:

N=total number of samples used; and
a and b correspond to the values compared.

The values calculated from equation 1 form a symmetric matrix of the form of equation 2.

$$S = \begin{matrix}{s}_{210 , 210} & {s}_{210 , 220} & {s}_{210 , 230} \\ {s}_{220 , 210} & {s}_{220 , 220} & {s}_{220 , 230} \\ {s}_{230 , 210} & {s}_{230 , 220} & {s}_{230 , 230}\end{matrix} \qquad ( 2 )$$

The values determined for the quantities of A_{B}, tc and I2Disp for all of the reference samples may be combined into vectors as shown in equation 3:

$${x}_{Sample} = [ \begin{matrix}\begin{matrix}{A}_{B} \\ tc\end{matrix} \\ I 2 Disp\end{matrix} ] \qquad ( 3 )$$

Each sample of the desired data set will provide one vector, x_{Sample}. Each vector will then be used in the calculation of equation 4 to produce one scalar, shown in equation 4 as D_{Reference}, for each data set used to create the matrix S.

D_{Reference}=x_{Sample}^{T}S⁻¹x_{Sample} (4)

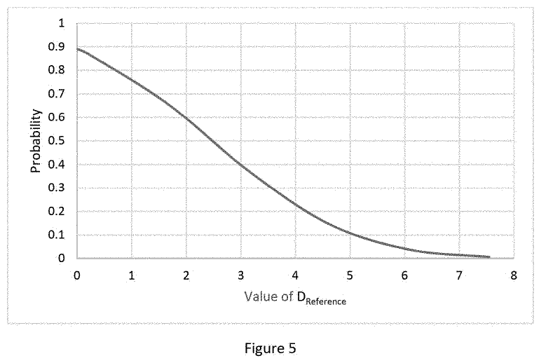

All of the values of D_{Reference} provide a distribution of the expected values of D_{Reference} for classification of future data sets obtained from the configuration of energy source controller 101; energy source 105; tested material 110; sensor 115; and receiver module 120. FIG. 5 shows an example of such a distribution. The distribution can be defined as a probability density function using the average and standard deviation as definition values for D_{Reference}.

The properties of the distribution; such as mean, standard deviation and the form of the distribution: the values in the matrix S⁻¹; and other definition values as may be relevant; are then stored by the analysis module 150 in the data storage module 135.

Consider a specific example where the quantities identified above are used. In this example, the energy source 105 is co-located with the sensor 115 to apply and receive ultrasonic energy from a specimen 110. For the reference samples, outputs from the attenuation inversion module 125 appear similar, but not identical, to FIG. 2. Thirty-five different reference samples have been used by the analysis module 150 to produce the matrix S⁻¹ which is stored in the data storage module 135. The matrix in this particular example is:

$${S}^{- 1} = [ \begin{matrix}2.444 & 5.527 & - 4.85 \\ 5.527 & 21.971 & - 19.91 \\ - 4.85 & - 19.91 & 19.096\end{matrix} ]$$

When the D_{Reference} values were calculated for all the reference samples, the distribution of the values can be represented by the chart shown in FIG. 5. The chart, or its mathematical representation can be used to determine the probability that any particular D_{Reference} One value is similar to the reference sample values. For example, in this case, acceptable readings could be identified if the D_{Reference} value is less than 2.5 which corresponds to a probability greater than 0.5. In some cases, determining the factors governing a particular decision may require input from a person knowledgeable in the particular non-destructive testing technique. After the factors are determined, the evaluation can be automated in order to reduce the amount of time and energy a skilled person would have to use. Further, with the evaluation being automated, more accurate results are expected to be received.

Other information may be placed in the data storage module 135. This includes parameters that may be used for various functions, including: determine what actions are to be taken with certain findings from the data sets; definitions for acceptance or rejection of data sets; equations and mathematical operations to be completed that may not be in the existing classification module; equations and algorithms that may be used to identify anomalies in data as knowledge of these develops with experience; and other data.

When the data as described above has been provided to the data storage module 135, the system in FIG. 1 may be used to evaluate data sets from other or unknown material tests.

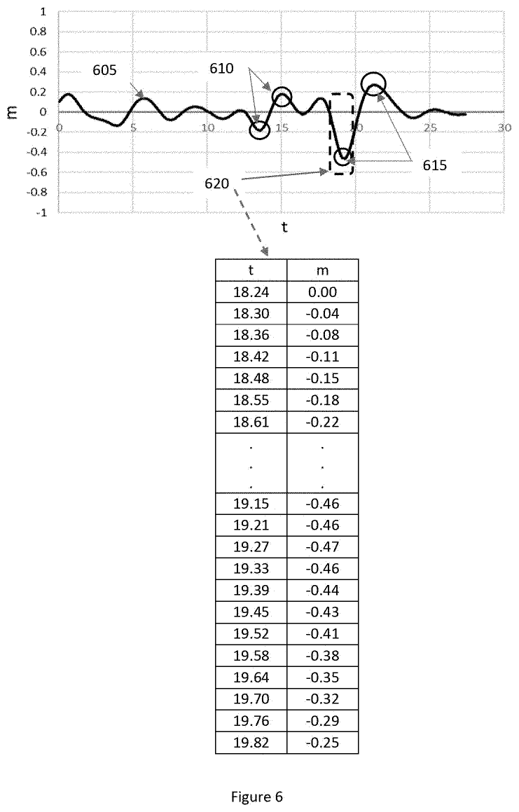

FIG. 6 shows an example of a data set 605 for a similar ultrasonic test from a test material 110 that may be received at the receiver module 120. In an interpretation, the circled areas 610 and 615 may correspond to energy that has been received by the sensor 115 from some physical feature within the material that has interrupted the energy transmission. The rectangular zone 620 may correspond to the same set of t values as 420. Individual coordinate values are provided in the chart of FIG. 6. Once this data set is received it may be transferred to the attenuation inversion module 125.

As detailed herein, the attenuation inversion module 125 will perform various programmed calculations and transformations to invert or cancel out the natural attenuation of energy that is expected by the material 110. For ultrasonic energy, it is well known that the attenuation can be reversed by applying a transformation of the form given in equation 5:

y=me^{At} (5)

### Where:

m=the magnitude value corresponding to t from the receiver module 120; and
A=a constant value; and
t=the value along the horizontal axis.

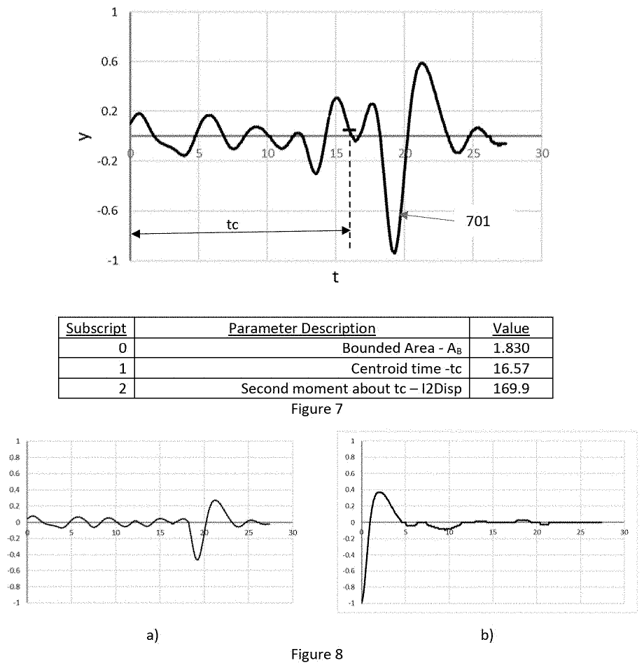

This transformation has been applied to the data set 605 and is shown in the graph in FIG. 7. The values for y in FIG. 4 are the result of the same transformation.

The result of this is shown graphically in FIG. 7 as 701, and also includes values for A_{B}, tc, and I2Disp similar to those shown in FIG. 4.

The result of the attenuation inversion module is transmitted to the classification module 130 which: forms the vector x as in equation 3; calculates the scalar D using equation 4; determines the probability value associated with D using the distribution in FIG. 5; compares the probability value with parameters such as acceptance and rejection criteria; determines data to be transmitted to the output module 140.

For this example, the calculations provided for by the classification module 130 are:
a. The values for A_{B}, tc, and I2Disp are calculated and the vector is formed;

$${x}_{example} = [ \begin{matrix}\begin{matrix}4.346 \\ 16.05\end{matrix} \\ 197.1\end{matrix} ]$$

b. Equation 4 is calculated to produce:

Dₑₓₐₘₚₗₑ=320.8
c. And FIG. 3 is used to determine the probability that the reading is similar:

Probability=0

These results show that the data provided for the chart in FIG. 7 is not similar to the reference samples, and the system obtaining this result would mark the data as “Not acceptable”. The results of the analysis will then be transferred to the output module 140 and recorded in the memory module 145.

The output module 140 will provide various outputs following instructions from classification module 130 and other instructions as may be provided by, for example, instructions previously stored in the data storage module 135. The outputs will identify acceptance or rejection of the data set. Other outputs may be: instruction to test the next material; instructions or direct transmission of the data set to another system; instructions to dispose of the tested material; identification of error conditions or faults in the evaluation; or other signals.

In some cases, the classification module 130 may be configured to calculate values for data patterns from each signal, assemble the values into a vector and calculate the likelihood that the features correspond to a known classification of sensor data. The classification module is configured to extract relevant data, as identified by programming and data storage module 135, from the sensor signal data/inverted signal data.

The matrix S⁻¹ is intended to provide benefit in determining how similar a reading was to the set of readings, by, for example, using equation 4 and the graph illustrated in FIG. 5. The set of similar readings that were used to develop S⁻¹ are considered to define a “Class” of readings. In the specific example, when the reading from the example in FIG. 7 was processed, the probability that it was a member of the defined class was 0 and it may be concluded that the example reading is not a member of the class.

FIGS. 8A and 8B show two example readings from a similar energy and sensor arrangement as FIG. 4. The locus shown in FIG. 8A differs noticeably from FIG. 8B. In fact, these readings could be considered to represent two different classes of readings and are designated as “Class a” for the reading from FIG. 8A and “Class b” from FIG. 8B.

Table 1 shows the results of the classification calculations using S⁻¹, equation 4 and FIG. 5 for the 2 readings.

TABLE 1

| Description | Reading in FIG. 8A | Reading in FIG. 8B |
|---|---|---|
| A_{B} | 1.830 | 2.00 |
| tc | 16.570 | 4.484 |
| I2Disp | 169.9 | 55.04 |
| D | 2.892 | 80.9 |
| Probability | 0.37 | 0 |

These calculations show that the reading in FIG. 8A is much more similar to the class than the reading in FIG. 8B.

The variation that occurs within a data set can be calculated as variance or standard deviation. When 30 or more values are used to calculate these, the addition of a lone value can alter the calculated variance by less than 3% and thus, a data set containing at least 30 values can be used to characterize the variation within the data set with a predetermined level of confidence. The number of values in the data set can be increased to a higher value if a greater level of confidence is desired.

Now consider that at least thirty (30) readings that are known to belong to the same class as FIG. 88, because they were obtained under the same general set of controlled conditions, are available. Values from these readings are provided to the analysis module 150 by the memory module 145. As above, the same values were determined for each reading and equation 1 was used to determine the matrix S⁻¹ that is specifically for this class. The elements in the matrix can be represented as:

$$[ \begin{matrix}1.105 & 0.211 & - 0.482 \\ 0.211 & 11.878 & - 11.403 \\ - 0.482 & - 11.403 & 12.048\end{matrix} ]$$

Note that the elements in this matrix are different from the values shown above for S⁻¹.

Table 2 shows the results for evaluation of the readings in FIGS. 8A and 8B using the new matrix determined for the class represented by FIG. 8B.

TABLE 2

| Description | Reading in FIG. 8A | Reading in FIG. 8B |
|---|---|---|
| A_{B} | 1.830 | 2.00 |
| tc | 16.570 | 4.484 |
| I2Disp | 169.9 | 55.04 |
| D | 293.662 | 2.381 |
| Probability | 0 | 0.52 |

The results show that the reading for 8B is similar to the class and FIG. 8A is not similar to the class.

Referring back to FIG. 1, the classification module may contain more than one set of calculations that may be used to identify the class that the data, provided by the attenuation inversion module 125, is best matched to. This may be done by including additional S⁻¹ matrices that relate to different classes of data or readings in data storage module 135 and to complete additional calculations in classification module 130 so that data can be further classified according to additional criteria that may be determined from time to time. Providing these additional calculations will allow data that may be acquired from external sources to be evaluated for different classes that may be useful or needed.

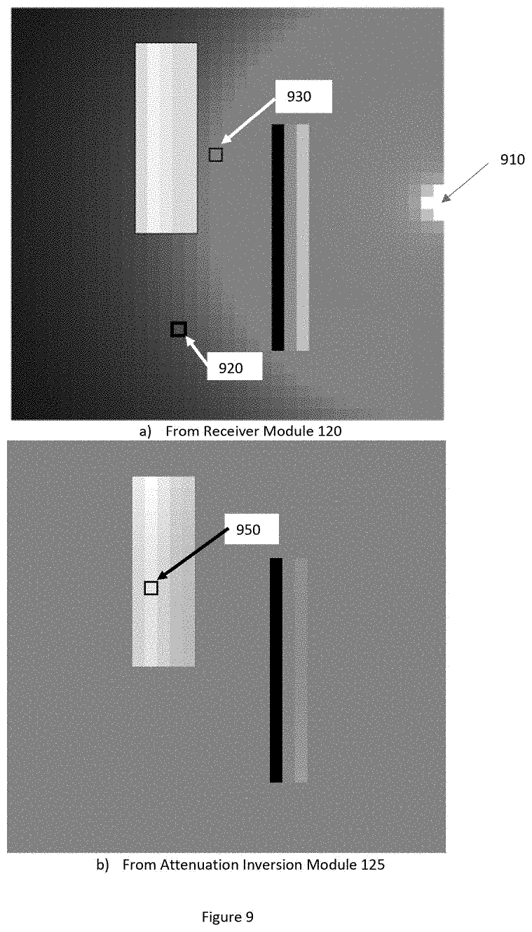

FIGS. 9A and 9B show two images such as may be received by a two-dimensional sensor array. Examples of two-dimensional sensor arrays may include: photographs; microscope images; thermographs; and others.

FIG. 9A shows an as-received image from a sensor array, which includes a plurality of individual sensors where an energy source 910 produces an image received by the system. Within the image, data values relate to the shading of individual elements of the image. For example, an area 920 has a value that has been detected.

For this image, the location of the centroid of the image area based on the data received from the sensor is marked as 930. The location of the centroid is an example of a characteristic that may be of interest.

FIG. 9B shows the image after the image has been adjusted to make the energy distribution by the energy source 910 uniform using an attenuation inversion by, for example, the attenuation module 125. It may be noted that the location of the centroid 950 has changed from the location of 930. For the system and method described above, this two-dimensional data may also be analyzed using other or similar automated methods as detailed herein and as known to determine characteristics.

In this manner, embodiments of the system and method may be extended to data sets of any number of dimensions and sensors.

In some cases, the system may further include a memory component configured to store sensor signal data and classification data. The sensor signal data and classification data may be used to determine trends or historic information if desired; provide updated or alternate calculations of the S matrix; provide a record of material tests; provide data for identification of other values that may be used for classification; and other purposes.

In some cases, the system may use the sensor data in its raw form as-received directly by the receiver. In other cases, the system may use the sensor data in a form that has been altered by transformations applied by elements of the system, such as inversion or the like. Selection of the use and acquisition of sensor data lies with the analysis of the underlying data by one sufficiently skilled in the art.

In a specific example, the at least one sensor may detect a series of readings. In this case, the classification module can be configured to use values calculated from the series of readings to classify the sensor signal data. This situation may occur where data is to be acquired from discrete locations that are sufficiently separated to allow data to be obtained as a series of readings. This method might also be used to reduce the number and cost of sensors,

The method and system described herein may be applied to data from a single sensor, or data from a plurality of sensors, or a series of data from a single sensor or a series of data from a plurality of sensors.

The method and system described herein may also apply to evaluation of data for a plurality of classifications from the same set of sensor data. For example, one set of sensor data could be evaluated within the same system to determine different classifications of data such as: material or component shape; and material or component thickness; and material or component position; and other characteristics that may be detectable in the same set of data.

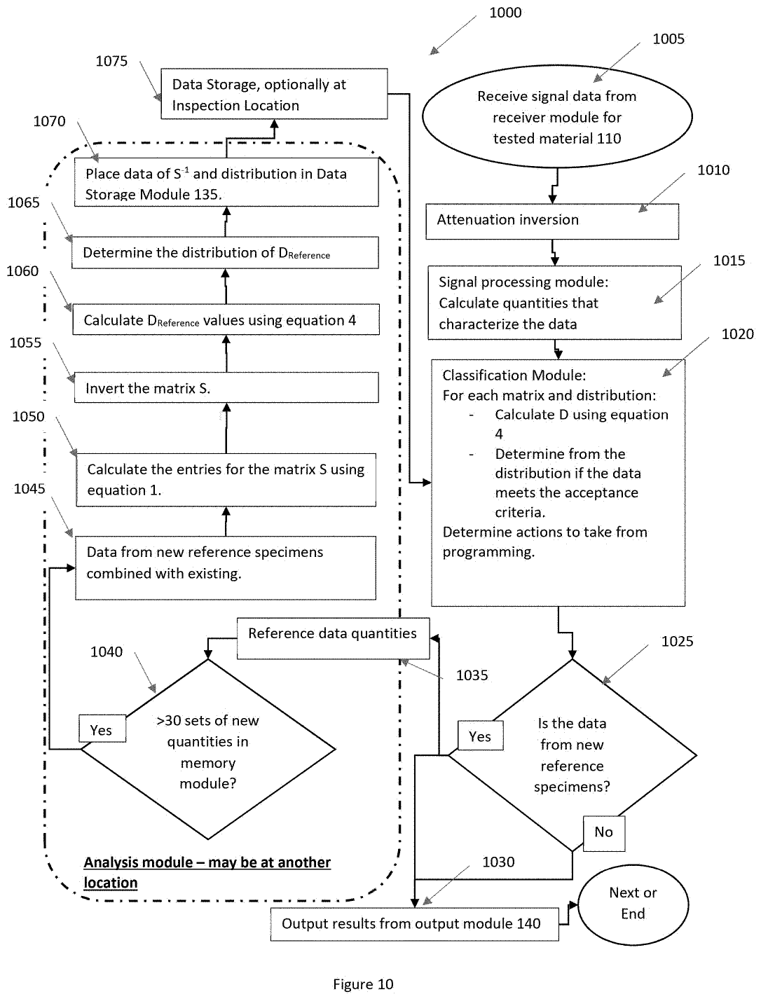

FIG. 10 illustrates a method 1000 for classification of sensor data according to an embodiment. Energy is provided to a material or component by a transmitter. At 1005, signal data is received from the receiver module 120 of a tested material or component 110. The signal data is intended to be sent from at least one senor that receives the transformed energy and converts the transformed energy into signal data. An attenuation inversion may then be applied to the received data, at 1010. At 1015, the analysis module may calculate quantities that characterize the data, such as, for example: the location of the centroid of the values from the centroid; the second moment of the values as they are distributed about the centroid; sums of values along particular directions; and other quantities that characterize the data depending on the application of the technology.

At 1020, the classification module 130 may then determine each matrix after retrieving further data from the data storage module 135. The classification may determine from the distribution retrieved from the data storage module, if the data meets the acceptance criteria. If the data is not from a new reference specimen, at 1025, the classification module may provide output to the output module, at 1030 and these results may be provided to the user or action associated with the material or component may be taken based on the output.

If the reference specimen is new, the system is configured to further define reference data quantities, at 1035. In a particular example, the analysis module 150 may require a predetermined number of sets of data prior to determining a classification model. In this case, the analysis module 150 may determine if there is a sufficient number of new quantities in the memory module 145, at 1040, or if further samples may be needed or useful (in this case 30 sets of new quantities is the threshold but this number can be configured depending on the application and the like). If there are sufficient samples, data from new reference specimens may be combined with previously determined specimens, at 1045.

At 1050, the entries of the matrix S may be calculated and the matrix may then be inverted, at 1055. D_{reference} may be determined from the matrix S⁻¹ using equation 4, at 1060. At 1065, the distribution of D_{reference} may be determined by the analysis module 150. At 1070, the data of the matrix S⁻¹ may be stored in the data storage module 135 as may be the distribution. At 1075, the data storage module is intended to be accessible at the inspection location of the material and may be fed to the classification module 130.

In the preceding description, for purposes of explanation, numerous details are set forth in order to provide a thorough understanding of the embodiments. However, it will be apparent to one skilled in the art that these specific details may not be required. It will also be understood that aspects of each embodiment may be used with other embodiments even if not specifically described therein. Further, some embodiments may include aspects that are not required for their operation but may be preferred in certain applications. In other instances, well-known structures may be shown in block diagram form in order not to obscure the understanding. For example, specific details are not provided as to whether the embodiments described herein are implemented as a software routine, hardware circuit, firmware, or a combination thereof.

Embodiments of the disclosure or elements thereof can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with other modules and elements, including circuitry or the like, to perform the described tasks.

The above-described embodiments are intended to be examples only. Alterations, modifications and variations can be effected to the particular embodiments by those of skill in the art without departing from the scope, which is defined solely by the claim appended hereto.

I claim:

1. A system to evaluate or classify non-destructive testing sensor data, the system comprising:
a transmitter configured to provide energy to a material;
one or more sensors configured to convert the energy returned from the material into sensor data;
a receiver configured to receive sensor data;
an attenuation inversion module configured to apply a mathematical transformation to the sensor data to provide transformed sensor data;
an analysis module configured to process the transformed sensor data to provided processed sensor date, by:
determining values from the transformed sensor data;
applying mathematical transformations to the values to produce a set of single values that represent the sensor data;
a classification module configured to classify the processed sensor data; and
an output module configured to output the results of the classification.

2. A system according to claim 1, further comprising a memory component configured to store the parameters to be used in the mathematical transformations used in the attenuation inversion module.

3. A system according to claim 1, further comprising a data storage module configured to store:
a distribution of known classes for the sensor data
the values determined from the processing module;
the results of classification; and
specific values for limits and boundaries on values determined from signal.

4. A system according to claim 1, further comprising a calculation component that determines parameters and transformations to define new classes.

5. A system according to claim 1, wherein data associated with the energy transmitter, the at least one sensor, and the receiver is included with the sensor data.

6. A system according to claim 3, wherein starting and ending coordinates is be applied to the sensor data to be evaluated as a value for the boundaries determined from the sensor.

7. A system according to claim 1, wherein the classification module is configured to determine the most probable class related to sensor data.

8. A system according to claim 1, wherein the classification module is configured to provide data relating to the transformation of values calculated from the sensor data.

9. A system according to claim 1, wherein the output module is configured to provide data related to whether sensor data is within a stated confidence interval for its classification.

10. A method to evaluate and classify sensor data, the method including:
applying energy to a material;
receiving transformed energy at sensors as signal data;
amplifying the signal data, in an attenuation inversion module;
processing the amplified signal data, at a signal processing module;
classifying processed signal data;
storing processed signal data related to the sensor signals in a data storage module together with the results of classification;
displaying the classification results, at an output module;
transferring stored data to a classifier module, wherein the data is used to calculate and update transformations and distributions to be applied to future data and;
storing the updated transformations and distributions in a data storage module.

11. A method according to claim 10, further comprising storing a plurality of possible classifications of the sensor data.

12. A method according to claim 10, wherein attenuation inversion calculations are applied to the sensor data.

13. A method according to claim 10, wherein the sensor data includes estimated physical dimensions of the material.

14. A method according to claim 10, further comprising applying limits and boundaries on values determined from the signal data and provided by the data storage module.

15. A method according to claim 15, further comprising using stored values for limits and boundaries on values determined from signal data and using starting and ending coordinates in the data set for the signal data to be evaluated.

16. A method according to claim 10, further comprising transforming the signal data into characteristic values using specific data provided by the data storage module.

17. A method according to claim 16, further comprising comparing characteristic values from transformation of the signal data to a known distribution.

18. A method according to claim 10, further comprising providing output related to the most probable class related to the sensor data.

19. A method according to claim 10, wherein the sensor data from materials that are confirmed to be reference samples are used to develop new transformations to be used in classification.

20. A method according to claim 19, wherein a new distribution of results is calculated based on the new transformations.
